(19) United States
(12) Patent Application Publication
(10) Pub. No.: US 2017/0260394 A1
(43) Pub. Date: Sep. 14, 2017

(54) A NEW PROCESS FOR PRODUCING GARDENIA BLUE PIGMENT

(71) Applicant: DSM IP Assets B.V., Heerlen (NL)

(72) Inventors: Qing HE, Shanghai (CN); Xiaoping HUANG, Shanghai (CN)

(21) Appl. No.: 15/511,468

(22) PCT Filed: Sep. 16, 2015
(86) PCT No.: PCT/CN2015/089795

§ 371 Date: Mar. 15, 2017

(30) Foreign Application Priority Data
Sep. 17, 2014 (CN) PCT/CN2014/086693

(51) Int. Cl. C09B 61/00; C12P 17/06; A23L 5/43; C09B 67/54

(52) U.S. Cl. CPC C09B 61/00; C09B 67/0096; C12P 17/06; A23L 5/43; A23V 2002/00

(57) ABSTRACT

A process for producing the gardenia blue pigment is provided. The process is easy to operate and suitable for industry and the obtained gardenia blue pigment is bright and suitable for industrial application.

### TECHNICAL FIELD

The present invention is related to a process for producing a natural pigment. In particular, the present invention is related to a process for producing the gardenia blue pigment.

### BACKGROUND OF THE INVENTION

The gardenia blue pigment is a water-soluble natural pigment widely used in food, pharmaceutical and cosmetics industries.

Nowadays, the gardenia blue pigment is usually produced from the raw material geniposide contained in Gardenia Jasminoides Ellis of Rubiaceae by the process of treating geniposide with aβ-glucosidase to obtain genipin which reacts with an amino acid to obtain the gardenia blue pigment. (see Shijing W U, et al., National Food Additive Communications, 1992 (3): 90-93) However, the gardenia blue pigment obtained by this process is dark, it has a low color value and is of low quality. So it is not suitable for some applications such as beverages. (see e.g. CN103509368A and CN102021210A)

Feng CHEN discloses a process for producing the gardenia blue pigment with a high color value, which comprises ultra-filtering the gardenia blue pigment obtained from the reaction of genipin with an amino acid to remove the residual geniposide and then extracting the filtrate to obtain the gardenia blue pigment of a high color value(see CN103525883A). Lijun SUN et al. discloses another process, which comprises passing the raw material geniposide through a large mesh non-polar resin to remove a-crocin before treating it with β-glucosidase. (Lijun SUN et al., Journal of Nanjing Agricultural University, 1994, 17 (4): 98-101) However, these processes involve high cost and complicated operations, and are not suitable for being carried out at large scale in industry.

Therefore, there is still the need for a new process, which is easy to operate and suitable for industry, for producing the gardenia blue pigment which is bright and suitable for industrial applications.

### SUMMARY OF THE INVENTION

The present invention provides a new process for producing the gardenia blue pigment, comprising the following steps:
a) Treating geniposide with a glycosidase to obtain a hydrolysate; and
b) Extracting the hydrolysate obtained in step a) with a solvent and removing the solvent after the extraction to obtain a product comprising genipin; and
c) Reacting the product comprising genipin obtained in step b) with an aqueous solution of an amino acid and/or a salt thereof to produce the gardenia blue pigment, and preferably dissolving the product comprising genipin in a water-soluble solvent resulting in a solution and using the solution as such; and
d) Optionally, purifying the gardenia blue pigment produced in step c).

The gardenia blue pigment obtained by the process of the present invention is sky blue, brighter than the blue such as ultramarine blue produced by the known processes. In addition, most of the obtained gardenia blue pigment has a color value of >100, so low dosage can be used in industrial applications.

### DETAILED DESCRIPTION OF THE INVENTION

The present invention provides a process for producing the gardenia blue pigment which is sky blue and may have a color value of >100. In particular, the process of the present invention comprises the following steps:
a) Treating geniposide with a glycosidase to obtain a hydrolysate; and
b) Extracting the hydrolysate obtained in step a) with a solvent and removing the solvent after the extraction to obtain a product comprising genipin; and
c) Reacting the product comprising genipin obtained in step b) with an aqueous solution of an amino acid and/or a salt thereof to produce the gardenia blue pigment, and preferably dissolving the product comprising genipin in a water-soluble solvent resulting in a solution and using the solution as such.

In the process, the geniposide used as raw material may be from various sources. It may be obtained by extracting the fruit Gardenia Jasminoides Ellis by any known process, for example, that as disclosed in Chinese patent publication CN102732050A. In addition, geniposide powders, which contains about 20 wt % to about 70 wt % of geniposide and is commercially available, and the waste stream from the gardenia yellow production, which contains about 40 wt % of geniposide and is also commercially available, may be used into the process directly or after simple refining. (see CN103509368A, CN103525883Aetc.)

The glycosidaseis anenzyme under EC 3.2.1 according to the Recommendations of the Nomenclature Committee of the International Union of Biochemistry and Molecular Biology on the Nomenclature and Classification of Enzymes by the Reactions they Catalyse. Preferably, the glycosidase is cellulase (EC 3.2.1.4) and 13-glucosidase (EC 3.2.1.21). The examples of the glycosidase include but are not limited to cellobiase commercially available from Sunson Biotechnology Co. Ltd. (Guangzhou, China), Cellulase 4000 commercially available from DSM (China) Ltd. (Shanghai, China), and Rapidase 0 commercially available from DSM (China) Ltd. (Shanghai, China).The glycosidase may be added into the reaction of the step a) in an amount in the range of from 0.01 g to 0.8 g, preferably in an amount in the range of from 0.05 g to 0.5 g, more preferably in an amount in the range of from 0.1 g to 0.3 g, per 1 g of geniposide.

The treatment of the step a) may be carried out at a pH in the range of from 3.0 to 6.5, preferably at a pH in the range of from 3.6 to 6.0, and more preferably at a pH in the range of from4.0 to 4.6. Preferably, the treatment of the step a) is carried out in a buffer solution which can provide the above pH ranges. Such buffer solution is known in the art, and the examples include but are not limited to an aqueous HCOOH/NaOAc solution or an aqueous citric acid/Na₂HPO₄ solution. The buffer solution may be added in an amount in the range of from 8 mL to 80 mL, preferably in an amount in the range of from 10 mL to 50 mL, more preferably in an amount in the range of from 20 mL to 40 mL, per 1 g of geniposide.

The treatment of the step a) may be carried out at a temperature in the range of from about 20° C. to about 60° C., preferably at a temperature of about 50° C.

The reaction of the step a) may last about 10 hours to about 30 hours. The obtained hydrolysate contains genipin as main component and other components. The hydrolysate can be used for the extraction in step b) directly.

In the prior process, the hydrolysate obtained from the step a) is normally reacted with an amino acid directly to produce gardenia blue pigment. However, the gardenia blue pigment obtained from the prior process is dark and not good for some industrial applications such as beverages. The inventors of the present invention surprisingly discovered that an additional extraction of the step b) results in sky blue color, a bright blue pigment which is more applicable for industrial applications like beverages.

Any person skilled in the art could understand that, the solvent used for extraction in the step b) is important and may be any one suitable for the purpose of the invention, and examples include but are not limited to diethyl ether, ethyl acetate, butanol, a mixture of butanol with petroleum and/or hexane, or mixtures thereof. In the embodiment that the solvent is a mixture of butanol with petroleum and/or hexane, the volume ratio of butanol with hexane and/or hexane is in the range of from 1:5 to 5:1, preferably in the range of from 1:3 to 3:1, and more preferably in the range of from 1:2 to 2:1.

The amount of the solvent used in the step b) may be from 1 mL to 5 mL, preferably from 1.5 mL to 3 mL, per 1 mL of the hydrolysate. The extraction of the step b) may be carried out at a temperature in the range of from 10° C. to 60° C., preferably at room temperature. According to the present invention, the extraction of the step b) may be repeated two to four times.

The product comprising genipinis obtained after the organic phases are collected and the solvent is removed in the step b).The solvent may also be recycled. The extraction procedures and the procedures for collecting solvents and removing/recycling solvents during the extraction are known to the person skilled in the art. Therefore, they are not discussed in more detail here.

As known in the art, the gardenia blue pigment is the reaction products of genipin with an amino acid or a salt thereof. Accordingly, as the step c) of the process, the product comprising genipin obtained in the step b) is reacted with an amino acid or a salt thereof to produce the gardenia blue. In the present invention, the amino acid suitable for the reaction may be selected from the group consisting of glutamate, phenylalanine, histidine, leucine, isoleucine, arginine and mixture thereof. The salt may be any alkali metal salt such as sodium salt. Preferably the salt issodium glutamate. The inventors of the present invention discovered that the amino acid and the salt used in the step c) are also important because they can provide sky blue color as disclosed in the present invention

In the present invention, an aqueous solution of the amino acid or the salt thereof is used to react with the product comprising genipin to provide the gardenia blue pigment and the aqueous solution is added in an amount to provide a molar ratio of 1:0.5-2, preferably 1:0.7-1.5 between genipin and the amino acid in the reaction mixture of step c).

In addition, the inventors of the present invention discovered that it would be helpful for the reaction in the step c) to dissolve the product comprising genipin in a water-soluble solvent to form a homogeneous reaction system. Accordingly, the product comprising genipin obtained from the step b) is preferably dissolved in a water-soluble solvent according to the present invention. The water-soluble solvent may be any one known in the art that can dissolve the product comprising genipin and examples include but are not limited to C₁₋₁₀ alkanols such as methanol and ethanol, and C₃₋₁₀ ketones such as acetone. Preferably, the water-soluble solvent is methanol, ethanol or acetone or any mixture thereof.

The reaction of the step c) may be carried out at about 40° C. to about 90° C., preferably about 60° C. to 80° C., such as 65° C., 70° C. and 75° C. The progress of the reaction can be monitored by any known method, such as HPLC and TLC.

Preferably, the step c) is carried out at a pH value in the range of from 7.0-11, more preferably 8-9.5. In some embodiments, a base selected from but not limited to NaOH, KOH, NaCO₃ and NaHCO₃ is added to adjust the reaction mixture of the step c) to an appropriate pH value.

After the reaction is complete, the gardenia blue pigment can be obtained as a solid by removing the organic solvent and water in the reaction mixture. Accordingly, the process of the present invention optionally further comprises the step of removing the solvent and water to provide a solid of the gardenia blue pigment by, for example, lyophilization or spray drying.

Optionally, the obtained gardenia blue pigment can be purified further by any procedures known in the art such as ultrafiltration to obtain an even purer gardenia blue pigment. The process of the present invention produces the gardenia blue pigment which is sky blue, brighter than the blue color such as ultramarine blue produced by the known processes and thus more popular for some industrial applications such as beverages.

In addition, most of the obtained gardenia blue pigment has a color value of >100, that means low dosage can be used in applications. Further, by an additional extraction step, the obtained gardenia blue pigments can surprisingly be easily separated and purified from the reaction mixture without complicated operations.

The present invention is illustrated further by the following Examples. These Examples are not intended to limit the invention in any way.

### EXAMPLES

In the following examples, the maximum absorption wavelength and the color value were measured according to the national standard GB 28311-2012 of China.

### Example 1

6.7 g of geniposide powder (35.5 wt %)purchased from Jiatian Biotechnology Co., Ltd. (Xi'an, China) was added into 47 ml of an aqueouscitric acid/Na₂HPO₄buffer solution (pH 4.0). 685.7 mg of cellobiase purchased from Sunson Biotechnology Co. Ltd. (Guangzhou, China) was further added for reaction for 19 hours at 50° C. After having cooled down the reaction mixture to room temperature, the reaction mixture was extracted with 90 ml of ethyl acetate twice. The ethyl acetate phase was concentrated under vacuum to obtain rude genipin.

The obtained rude genipin was dissolved in 16 ml of absolute ethyl alcohol to obtain 19.4 g of a solution (4.79 wt % genipin by HPLC). 970 mg of sodium glutamate in 10 ml of deionized water was added into the solution for reaction for 25 hours at 70° C. TLC indicated that the genipin had been converted completely.

The reaction liquid was lyophilized to obtain 2.1 g of gardenia blue pigment as solid powder, with a maximum absorption wavelength of 601 nm and a color value of 155.

### Example 2

20g of waster of gardenia yellow (40 wt % geniposide) purchased from Qianjiang Green Sea

Treasury Biotechnology Co., Ltd. (Hubei, China) was added into 140 ml of an aqueous citric acid/Na₂HPO₄buffer solution (pH 4.6). 800 mg of Rapidase 0 purchased from DSM (China) Ltd. (Shanghai, China) was further added for reaction for 16 hours at 50° C. After having cooled down the reaction mixture to room temperature, the reaction mixture was extracted with 250 ml of ethyl acetate twice. The ethyl acetate phase was concentrated under vacuum to obtain rude genipin.

The obtained rude genipin was dissolved in 30 ml of absolute ethyl alcohol to obtain 34.8 g of a solution (8.4 wt % of genipin by HPLC). Into two reaction flasks with 4.0 g of the solution, 299.6 mg of histidine in 8 ml of deionized water and 310.5 mg of arginine in 6 ml of deionized water were added respectively for reaction for 30 hours at 70° C.

The reaction liquids were lyophilized to obtain gardenia blue pigment as solid powder, with maximum absorption wavelength and a color value as indicated in Table 1.

TABLE 1

| | gardenia blue pigment | | |
|---|---|---|---|
| | | Maximum | |
| Amino acid | Weight (g) | absorption wavelength (nm) | Color value |
| Histidine | 0.8 | 595 | 100 |
| Arginine | 0.6 | 597 | 127 |

### Example 3

8g of waster of gardenia yellow (40 wt % geniposide) purchased from Qianjiang Green Sea Treasury Biotechnology Co., Ltd. (Hubei, China) was added into 64 ml of an aqueous citric acid/Na₂HPO₄buffer solution (pH 4.5). 320 mg of Rapidase® purchased from DSM (China) Ltd. (Shanghai, China) was further added for reaction for 17 hours at 50° C. After having cooled down the reaction mixture to room temperature, the reaction mixture was extracted with 100 ml of diethyl ethertwice. The diethyl etherphase was concentrated under vacuum to obtain rude genipin.

The obtained rude genipin was dissolved in 28 ml of absolute ethyl alcohol to obtain 30.1 g of a solution (4.10 wt % of genipin by HPLC). Into a reaction flask with 6 g of the solution, 185.4 mg of isoleucine in 16 ml of deionized water was added for reaction for 33 hours at 65° C.

The reaction liquid was lyophilized to obtain 420 mg of gardenia blue pigment as solid powder, with a maximum absorption wavelength of 600 nm and a color value of 87.

### Example 4

10g of waster of gardenia yellow (40 wt % geniposide) purchased from Qianjiang Green Sea Treasury Biotechnology Co., Ltd. (Hubei, China) was added into 70 ml of an aqueous citric acid/Na₂HPO₄ buffer solution (pH 4.6). 400 mg of Rapidase® purchased from DSM (China) Ltd. (Shanghai, China) was further added for reaction for 30 hours at 50° C. After having cooled down the reaction mixture to room temperature, the reaction mixture was extracted with 120 ml of ethyl acetate twice. The ethyl acetate phase was concentrated under vacuum to obtain rude genipin.

The obtained rude genipin was dissolved in 16 ml of absolute ethyl alcohol to obtain 15.02 g of solution (10.62 wt % of genipin by HPLC). 1.31 g of sodium glutamate in 10 ml of deionized water was added into the solution for reaction for 33 hours at 65° C.

The reaction liquid was lyophilized to obtain 3.0g of gardenia blue pigment as solid powder, with a maximum absorption wavelength of 598 nm and a color value of 132.6.

### Example 5

10 g of waster of gardenia yellow (40 wt % geniposide) purchased from Qianjiang Green Sea Treasury Biotechnology Co., Ltd. (Hubei, China) was added into 70m1 of an aqueous citric acid/Na₂HPO₄ buffer solution (pH 4.6). 2 g of cellobiase purchased from Sunson Biotechnology Co. Ltd. (Guangzhou, China) was further added for reaction for 30 hours at 50° C. After having cooled down the reaction mixture to room temperature, the reaction mixture was extracted with 120 ml of ethyl acetate twice. The ethyl acetate phase was concentrated under vacuum to obtain rude genipin.

The obtained rude genipin was dissolved in 12 ml of absolute ethyl alcohol to obtain 11.95 g of solution (12.38 wt % of genipin by HPLC). 1.21 g of sodium glutamate in 10 ml of deionized water was added into the solution for reaction for 33 hours at 65° C.

The reaction liquid was lyophilized to obtain 2.9 g of gardenia blue pigment as solid powder, with a maximum absorption wavelength of 594 nm and a color value of 143.9.

### Example 6

10 g of waster of gardenia yellow (40 wt % geniposide) purchased from Qianjiang Green Sea

Treasury Biotechnology Co., Ltd. (Hubei, China) was added into 70 ml of an aqueous citric acid/Na₂HPO₄ buffer solution (pH 4.0). 1.2g of cellobiase purchased from Sunson Biotechnology Co. Ltd. (NingXia, China) was further added for reaction for 15 hours at 50° C. After having cooled down the reaction mixture to room temperature, the reaction mixture was extracted with 100 ml mixture of 2/1 butanol/petroleum for 3 times. The organic phase was concentrated under vacuum to obtain rude genipin.

The obtained rude genipin was dissolved in 30 ml of absolute ethyl alcohol to obtain 26.0 g of a solution (6.5 wt % of genipin by HPLC). 1.77 g of sodium glutamate in 30 ml of deionized water was added into the solution for reaction for 23 hours at 75° C. TLC indicated that the genipin had been converted completely.

The reaction liquids were lyophilized to obtain 2.8 g gardenia blue pigment as solid powder, with maximum absorption wavelength of 594 nm and a color value of 140.

### Example 7

40g of waster of gardenia yellow (40 wt % geniposide) purchased from Henan Zhongda Hengyuan Biotechnology Co., Ltd. (Henan, China) was added into 280 ml of an aqueous citric acid/Na₂HPO₄ buffer solution (pH 4.0). 4.6 g of cellobiase purchased from Sunson Biotechnology Co. Ltd. (NingXia, China) was further added for reaction for 19 hours at 50° C. After having cooled down the reaction mixture to room temperature, the reaction mixture was extracted with 300 ml ethyl acetate for 3 times. The organic phase was concentrated under vacuum to obtain rude genipin (70.8% purity, QNMR).

1 ml of ethanol were added to dissolve 50 mg of the above crude genipin solid in a flask. 116.4 mg of Phenylalanine and 92.4 mg of Leucine were dissolved and adjusted to pH9.2 and pH9.63 with 0.1N NaOH and then was charged into the flask for reaction for about 5 h at 80° C., respectively.

The reaction liquids were lyophilized to obtain gardenia blue pigment as solid powder, with maximum absorption wavelength and a color value as indicated in below Table 2.

TABLE 2

| | Gardenia blue pigment | | |
|---|---|---|---|
| | | Maxim | |
| Amino acid | Weight (mg) | absorption wavelength (nm) | Color value |
| Phe | 86.6 | 596 | 170.9 |
| Leu | 73.9 | 596 | 176.6 |

### Example 8—Comparative

8 g of geniposide powder (60 wt % geniposide) purchased from YuanYang Bio-engineering Co., Ltd.(Xi'an, China) was added into 64 ml of an aqueous citric acid/Na₂HPO₄ buffer solution (pH 4.6). 240 mg of Cellulase 4000 purchased from DSM (China) Ltd. (Shanghai, China)was further added for reaction for 24 hours at 50° C. Then 2.0 g of sodium glutamate was added into the reaction mixture for 96 hours at 50° C.

The reaction mixture was filtered, purified by ultrafiltration, and then lyophilized to obtain 2.8 g of gardenia blue pigment as solid powder, with a maximum absorption wavelength of 591 nm and a color value of 11.25.

### Example 9—Comparative

10g of waster of gardenia yellow (40 wt % geniposide) purchased from Qianjiang Green Sea Treasury Biotechnology Co., Ltd. (Hubei, China) was added into 70 ml of an aqueous citric acid/Na₂HPO₄ buffer solution (pH 4.5). 400 mg of Rapidase® purchased from DSM (China) Ltd. (Shanghai, China) was further added for reaction for 18 hours at 50° C. After cooled down the reaction mixture to room temperature, the reaction mixture was extracted with 100 ml of ether twice. The ether phase was concentrated under vacuum to obtain rude genipin.

The obtained rude genipin was dissolved in 28 ml of absolute ethyl alcohol to obtain 30 g of a solution (3.67 wt % genipin by HPLC), 741 mg of valine in 15 ml of deionized water was added into the solution for reaction for 35 hours at 70° C.

The reaction liquid was lyophilized to obtain 1.8 g of gardenia blue pigment as solid powder, with maximum absorption wavelength of 590 nm and color value of 104.

### Example 10

The obtained gardenia blues obtained from above examples 1-7 and the comparative examples 8-9 were compared. According to the Industrial International Standard Color Chart (“IISCC”), the gardenia blue pigments obtained from examples 1-7 are called “sky blue” while the gardenia blue pigment obtained from the comparative examples 8-9 is called “ultramarine blue”. Table 3 indicates the results. Obviously, the “sky blue” is brighter than the “ultramarine blue”.

1. A process for the preparation of gardenia blue pigment, comprising the following steps:
a) Treating geniposide with a glycosidase to obtain a hydrolysate; and
b) Extracting the hydrolysate obtained in step a) with a solvent and removing the solvent after the extraction to obtain a product comprising genipin; and
c) Reacting the product comprising genipin obtained in step b) with an aqueous solution of an amino acid and/or a salt thereof to produce the gardenia blue pigment, and preferably dissolving the product comprising genipin in a water-soluble solvent resulting in a solution and using the solution as such; and
d) Optionally, purifying the gardenia blue pigment produced in step c).

2. The process of claim 1, wherein the glycosidase is cellulase (EC 3.2.1.4) or β-glucosidase (EC 3.2.1.21).

3. The process according to claim 1, wherein the glycosidase is added into the reaction of the step a) in an amount in the range of from 0.01 g to 0.8 g, preferably in an amount in the range of from 0.05 g to 0.5 g, more preferably in an amount in the range of from 0.1 g to 0.3 g, per 1 g of geniposide.

4. The process according to claim 1, wherein the treatment of step a) is carried out at a pH in the range of from 3.0 to 6.5, preferably at a pH in the range of from 3.6 to 6.0, and more preferably at a pH in the range of from 4.0 to 4.6.

5. The process according to claim 1, wherein the solvent used in the step b) is diethyl ether, ethyl acetate, butanol, a mixture of butanol with petroleum and/or hexane, or mixtures thereof

6. The process according to claim 1, wherein the solvent is used in an amount in the range of from 1 mL to 5 mL, preferably from 1.5 mL to 3 mL, per 1 mL of the hydrolysate.

7. The process according to claim 1, wherein the amino acid used in step c) is selected from the group consisting of glutamate, phenylalanine, histidine, leucine, isoleucine, arginine and mixture thereof

8. The process according to claim 1, wherein the salt is alkali metal salt such as sodium salt, preferably the salt is sodium glutamate.

9. The process according to claim 1, wherein the product comprising genipin obtained from step b) is dissolved in a water-soluble solvent.

10. The gardenia blue pigments obtainable from the process according to claim 1.
